(19) United States
(12) Patent Application Publication
(10) Pub. No.: US 2020/0318137 A1
(43) Pub. Date: Oct. 8, 2020

(54) TRAIL-SECRETING MESENCHYMAL STEM CELLS AND USE THEREOF TO TREAT BRAIN TUMORS

(71) Applicant: SHENZHEN BEIKE BIOTECHNOLOGY CO., LTD., Shenzhen (CN)

(72) Inventors: Xiang HU, Shenzhen (CN); Wai Sang POON, Shenzhen (CN); Gang LU, Shenzhen (CN); Muyun LIU, Shenzhen (CN); Mingkai XU, Shenzhen (CN); Xianwei SU, Shenzhen (CN)

(21) Appl. No.: 16/089,992

(22) PCT Filed: Mar. 30, 2017
(86) PCT No.: PCT/CN2017/078684

§ 371 Date: Sep. 28, 2018

(30) Foreign Application Priority Data
Mar. 30, 2016 (CN) 201610194781.1
Mar. 24, 2017 (CN) 201710184390.6

(51) Int. Cl. C12N 15/86; C12N 5/10

(52) U.S. Cl. CPC C12N 15/86; C12N 2740/15043; C12N 2740/15032; C12N 5/10

(57) ABSTRACT

The invention relates to the field of genetic recombination and stein cell application. In particular, the invention provides a construct for expressing a soluble fragment of a secretory TRAIL, and a lentiviral expression vector comprising the construct. The present invention also provides a mesenchymal stein cell in which the construct is integrated into the genome, which can express and secrete the TRAIL fragment. The invention also provides the use of the construct or vector or mesenchymal stein cells for the treatment of brain tumors.

The present application claims priority to Chinese Patent Application No. 201610194781.1, entitled “Trail-Secreting Mesenchymal Stem Cells and Use Thereof to Treat Brain Tumors” filed on Mar. 30, 2016, and Chinese Patent Application No. 201710184390.6, entitled “Trail-Secreting Mesenchymal Stem Cells and Use Thereof to Treat Brain Tumors”, filed on Mar. 24, 2017, which are hereby incorporated into the present application by reference in their entirety.

### FIELD OF THE INVENTIONS

The invention relates to the field of genetic recombination and stein cell application. In particular, the invention provides a construct for expressing a soluble fragment of a secreted TRAIL, and a lentiviral expression vector comprising the construct. The present invention also provides a mesenchymal stein cell in which the construct is integrated into the genome, which can express and secrete the TRAIL fragment. The invention also provides the use of the construct or vector or mesenchymal stein cells for the treatment of brain tumors.

### BACKGROUND OF THE INVENTION

Tumor necrosis factor-related apoptosis-inducing ligand (TRAIL) is a member of the tumor necrosis factor superfamily. TRAIL initiates an exogenous apoptotic pathway by binding to cell death receptors (DR4 and DR5) and induces apoptosis in tumor cells. Moreover, the apoptosis-inducing activity is mainly directed against tumor cells, and does not have a killing effect on normal cells, and thus has certain tumor specificity. The most economical way to obtain TRAIL is genetic engineering expression and purification. Currently, the commonly used expression system includes E. coli prokaryotic expression system and cellular eukaryotic expression system. Since TRAIL is of mammalian origin, eukaryotic cell expression systems have better compatibility.

There are three problems in the process of eukaryotic expression of TRAIL 1. Secretion of TRAIL: the general eukaryotic expression vector lacks an effective secretory signal peptide sequence, and cannot transfer the expressed target protein to the extracellular medium but accumulates inside the cell, which on one hand affects the accumulation of target protein, and on the other hand, makes it difficult to the subsequent separation and purification process. 2. TRAIL solubility: The amino terminus of TRAIL molecule is a hydrophobic membrane transmembrane structure, which includes a large number of hydrophobic amino acid residues, resulting in poor water solubility of TRAIL, which tends to accumulate in solutions and lose activity. 3. Gene stability of the genetically engineered cell: common eukaryotic cell expression vector, after bringing the target gene fragment into the host cell genome, the target gene fragment cannot stably existes, and the target gene will be gradually lost as the host cell passes through the process of division, therefore constantly antibiotic selection and monoclonal cell screening are needed for maintaining the target gene fragment in the transfected cells.

There remains a need in the art to obtain constructs, vectors and host cells that are capable of stably and efficiently expressing and secreting active TRAIL or fragments. There is a further need in the art for constructs, vectors and host cells that are capable of treating diseases and for the preparation of related drugs that are capable of stably and efficiently expressing and secreting active TRAIL or fragments thereof.

### SUMMARY OF THE INVENTION

The present invention has been made in view of the above problems, and provides a mesenchymal stein cell in which an exogenous nucleic acid is integrated on its genome, which stably and efficiently expresses and secretes a TRAIL fragment. To this end, the present invention also constructs a lentiviral expression vector capable of expressing a soluble fragment of a secreted TRAIL, which can stably transfects a mammalian host cell and secretes the soluble fragment of the expressed TRAIL into the culture medium of the host cell, facilitates subsequent collection, isolation and purification of the fragment of the expressed TRAIL. The invention also provides the use of the mesenchymal stein cells for the treatment of a TRAIL-related disease or for the preparation of a medicament, including cancer or a tumor, in particular a brain tumor.

Particularly, the present invention provides an isolated construct, wherein the construct comprises (1) a secretory signal peptide encoding region; (2) a TRAIL trimer stable structure encoding region; and (3) a TRAIL fragment encoding region,

Wherein the TRAIL fragment has an amino acid sequence identical or substantially identical to the amino acid sequence of (a) or (b) below:

(a) amino acid residues 114 to 281 of TRAIL;

(b) having at least 90% identity, more preferably at least 95% identity, such as at least 96% identity, at least 97% identity, at least 98% identity, or at least 99% identity with the amino acid sequence of (a), and having TRAIL activity.

The secretory signal peptide of eukaryotes is present on the protein to be secreted or to become a transmembrane part, usually at the N-terminus of the protein. The signal peptide in the present invention refers to be a heterologous signal peptide, i.e., a signal peptide that is not naturally operably linked to a protein or polypeptide. The use of a signal peptide allows the target protein or polypeptide to be secreted to the extracellular area of the cells producing it or increases secretion of the protein. Commonly used signal peptides include fibrillin secretory signal peptide, growth factor signal peptide, hormone signal peptide, cytokine signal peptide and immune protein signal peptide. Examples of signal peptides include GDF signal peptide, IGF signal peptide, BMP signal peptide, neurotrophin signal peptide, PDGF signal peptide and EGF signal peptide, and hormone signal peptides (eg, growth hormone, insulin, ADH, LH, FSH, ACTH, MSH), TSH), or an interleukin signal peptide. Most of these signal peptides are derived from mammalian sources, such as human, mouse, rat, pig, monkey, etc. Preferably, the signal peptides used in the present invention are of mouse or human origin. The signal peptide can also be modified to enhance its ability to aid protein secretion.

The secretory signal peptide used in the construct of the present invention may be selected from the group consisting of human fibrillin secretory signal peptide, a human growth hormone secretory signal peptide, a human immunoglobulin signal peptide, a human interleukin 2 signal peptide, and the like.

The secretory signal peptide preferably used in the present invention is a secretory signal peptide derived from human fibrillin. Human fibrillin-1, or human fibrillin, is reported in Sakai L Y, Keene D R, Engvall E et al., 1986, J. Cell Biol. 103, “Fibrillin, a new 350-kD glycoprotein, is a component Of extracellular microfibrils”. Human fibrillin is a glycoprotein secreted by fibroblasts into the extracellular matrix. The human fibrillin secretory signal peptide has the following amino acid sequence: MRRGRLLEIALGFTVLLASYTSHGADA. Nucleic acid sequences encoding human fibrin secretory signal peptides can be used in the constructs of the invention. In the construct of the present invention, the secretory signal peptide encoding region preferably has the sequence as shown in SEQ ID NO: 2.

In the constructs of the invention, said TRAIL trimer stabilizing structure is used to help forming a stable trimer form of TRAIL or a TRAIL fragment. The trimer form of the TRAIL or TRAIL fragment is more stable and more active than the monomer. Trimer stabilizing structure useful in the present invention include isoleucine zipper structures, leucine zipper structures, and the like.

It is known in the art that a leucine zipper domain or an isoleucine zipper structure is found in a variety of natural proteins. The Leucine zippers structure refers to a peptide chain wherein there is a leucine residue in very 7 amino acid residues, and the helix formed by this peptide chain presents a hydrophobic surface formed by the leucine residues and a hydrophilic surface formed by the hydrophilic amino acid residues. The hydrophobic surface composed of leucine residues is a leucine zipper strip, and two peptides with said leucine zipper strip can form a dimer or a multimer such as a trimer by hydrophobic interaction. A leucine zipper domain or an isoleucine zipper structure can interact to form a dimer or trimer. The leucine zipper domain or the isoleucine zipper structure contained in the construct of the present invention can promote oligomerization of the fusion protein and thus can increase stability. Suitable human leucine zipper domains are, for example, the leucine zipper domains contained in the human c-fos, c-jun, c-myc, max and mdx1 proteins. In the present invention, leucine zipper or isoleucine zipper structural sequence is optimized and experimentally confirmed to allow the expressed TRAIL fragment to form a trimer. The inventors obtained an optimized nucleic acid sequence encoding an isoleucine zipper structure sequence by design and testing. In the construct of the present invention, a preferred TRAIL trimer stabilizing structure encoding region has the sequence as shown in SEQ ID NO: 3.

In the construct of the present invention, the TRAIL fragment is a fragment of amino acids 114-281 of the amino acid sequence as shown in SEQ ID NO: 5.

Human TRAIL is known in the art to be a type II transmembrane protein consisting of 281 amino acids. Wiley, S. R. et al., reported in Identification and Characterization of a New Member of the TNF Family that Induces Apoptosis. Immunity 3, 673-682 (1995). The amino acid sequence of the TRAIL is set forth in SEQ ID NO: 5.

The protein encoded by the TRAIL encoding region comprised in the construct of the present invention can be a fragment of the active TRAIL, i.e., a contiguous portion of the 281 amino acid amino acid sequence of TRAIL, which has activity of the TRAIL. Preferred TRAIL fragments of the invention include, for example, a fragment of number 114-281 amino acid residues of TRAIL sequence as shown in SEQ ID NO: 5. It is known in the art that the intracellular N-terminus of TRAIL has no signal peptide and the active portion is located in its extracellular portion, which comprises its 114 to 281 amino acid residues. The 114-281 amino acid residues of human TRAIL can also form trimer, which has zinc binding sites near the top thereof that play an important role in maintaining TRAIL activity and stability.

The protein encoded by the TRAIL encoding region comprised by the construct of the present invention may also have an amino acid sequence substantially identical to the 114-281 fragment of TRAIL. As used herein, “substantially identical amino acid sequence” refers to an amino acid sequence of a protein comprising substitution, deletion, addition or insertion of one to several (eg, 2, 3, 4 or 5) amino acids, which has identical, similar or better activity in comparison with the protein with the original amino acid sequence.

The construct of the present invention can be prepared by using conventional DNA synthesis technology. The construct can be inserted into an expression vector by conventional genetic engineering methods; a host cell can be transformed with the resulting recombinant expression vector; the resulting transformant can be cultured; and the polypeptide can be collected from the culture. This can be accomplished, for example, by the method described in Molecular Cloning, T. Maniatis et al, CSH Laboratory (1983).

The invention also provides a vector comprising the construct described above. The vector of the present invention may be a plasmid, a bacteriophage, a virus or the like, and is capable of independently replicating and having a selection marker in a genetically engineered host capable of expressing a protein. The vector is amplified and expressed upon entry into the host cell. Suitable genetically engineered hosts are well known in the art and can be E. coli, yeast, insect cells, animal cells, and the like. Preferably, the vector of the present invention is a vector expressed in an animal cell, such as a non-viral vector, a baculovirus expression vector, an adenovirus vector, a retroviral vector, a lentiviral vector. The vector which can be used as the present invention is preferably a lentiviral vector such as a lentiviral vector pCDH, in particular, a pCDH having the aforementioned construct of the present invention. In one of the examples of the present invention, the present invention provides pCDH-seTRAIL as prepared as shown in the Examples.

The present invention also provides a cell, particularly a mammalian cell, such as a human, mouse, rat, porcine, or monkey cell, whose genome is integrated with the above-described construct of the present invention, and expressing and secreting the TRAIL fragment.

The mammalian cells of the present invention described above may be stein cells, lymphocytes, T cells, B cells, macrophages, fibroblasts, tumor cells, and the like. Preferably, the present invention provides a stein cell, such as a mesenchymal stein cell, which integrates the aforementioned construct of the present invention on its genome and which expresses and secretes a TRAIL fragment.

Mesenchymal stein cell (MSC) is an important member of the adult stein cell family. MSC is derived from the mesoderm and ectoderm in early development. Mesenchymal stein cells have the characteristics, including multi-directional differentiation, supporting hematopoiesis, promotion of stein cell implantation, immune regulation and self-replication. Studies have found that mesenchymal stein cells can differentiate into various tissue cells such as fat, bone, cartilage, muscle, tendon, ligament, nerve, liver, heart muscle, endothelium, etc. under continuous induction conditions in vivo or in vitro, and can maintain multi-directional differentiation potential after successively subculture and freezing preservation. According to its source, mesenchymal stein cells can be classified into umbilical cord mesenchymal stein cells, bone marrow mesenchymal stein cells, and adipose-derived mesenchymal stein cells.

In one aspect of the present invention, the mammalian cell whose genome is integrated with the above-described construct of the present invention and expressing and secreting the TRAIL fragment is prepared by the method described below:

a. providing a construct as above-described, which comprising: (1) a secretory signal peptide encoding region; (2) a TRAIL trimer stable structure encoding region; and (3) a TRAIL fragment encoding region,

Wherein the TRAIL fragment has an amino acid sequence identical or substantially identical to the amino acid sequence of (a) or (b) below:

(a) amino acid residues 114 to 281 of TRAIL;

(b) having at least 90% identity, more preferably at least 95% identity, such as at least 99% identity with the amino acid sequence of (a), and having TRAIL activity;

b. Preparing a vector comprising the construct, preferably, the vector is an animal cell expression vector, preferably a lentiviral vector, such as pCDH;

c. Infecting a mammalian cell with the vector, which is preferably a stein cell, such as a mesenchymal stein cell.

The invention also provides a method for producing a TRAIL, the method comprising transforming a mammalian cell with the vector described above, expressing a TRAIL in the mammalian cell, the TRAIL being secreted into a culture outside the host cell.

The present invention provides a pharmaceutical composition for treating a disease associated with TRAIL, which comprises any of the above-described constructs or vectors or cells of the present invention.

The present invention provides the use of any of the above constructs or vectors or cells or pharmaceutical compositions of the present invention for the treatment of a disease associated with TRAIL. The present invention also provides the use of any of the above-described constructs or vectors or cells or pharmaceutical compositions of the present invention for manufacturing a medicament for treating a disease associated with TRAIL.

It is known in the art that TRAIL induces an apoptotic response through both DR4 and DR5 receptors. Thus, any of the constructs or vectors or cells or pharmaceutical compositions of the present invention can be used to treat a disease with a DR4 or DR5 receptor on a target cell.

Diseases treatable by the present invention include cancers and tumors such as brain cancer. The brain tumors treatable by the present invention include glioma and meningioma and the like. Glioma includes glioblastoma, anaplastic astrocytoma, gliosarcoma, anaplastic oligodendroglioma, degenerative ganglioglioma, pineoblasoma, medullobastoma, and the like.

### BRIEF DESCRIPTION OF THE DRAWINGS

FIG. 1 is an agarose gel electrophoresis pattern of a secretory TRAIL soluble fragment encoding gene recovered after double-enzyme digestion. On the right is the DNA band of interest, and on the left is Invitrogen's 1 Kb Plus DNA ladder molecular marker. The target nucleic acid fragment is approximately 711 bps.

FIG. 2 Electrophoresis map of lentiviral expression vector pCDH plasmid DNA recovered after double enzymes digestion. On the left is the DNA band of interest, and on the right is the Invitrogen 1 Kb Plus DNA ladder molecular marker. The target nucleic acid fragment is approximately 8,000 bps.

FIG. 3. Fluorescence micrograph of HEK293 cells co-transfected with lentivirus-package plasmids for 48 hours. GFP is a photo under fluorescence, Light is a white light photo in the same field of view, and Merge is an integrated photo of two fields of view.

FIG. 4. Fluorescence and white light micrographs of MSC cells infected with lentiviral particles. GFP is a photo under fluorescence, Light is a white light photo in the same field of view, and Merge is an integrated photo of two fields of view.

FIG. 5 shows an ELISA standard curve for detecting TRAIL. The abscissa is the absorbance at 450 nm and the ordinate is the TRAIL standard concentration (pg/ml).

FIG. 6 MTS assay for the growth inhibition of U87 tumor cells by culture medium supernatant of MSC cells infected with lentivirus.

FIG. 7. Stability of TRAIL secreted by MSC cells infected with lentiviral vectors.

FIG. 8 MSC cells infected with lentiviral vectors inhibit U87 solid tumors in mice.

### DETAILED DESCRIPTION OF THE PREFERRED EMBODIMENTS

### EXAMPLE 1

### Preparation of a Secretory-Type Construct Encoding a Secretory Fragment of TRAIL

(1) Synthesis of a double-strand nucleotide sequence having a sequence as shown in SEQ ID NO : 4

SEQ ID NO: 4:
(SEQ ID NO: 4)
tctagagccatgggtcgtcgagggcgtctgctggagatcgccctgggatt
taccgtgcattttagcgtcctacacgagccatggggcggacgcccgtatg
aaacagatcgaagacaaaattgaggagatccttagcaagatttaccatat
agaaaacgagatcgctcgtattaaaaagcttatcggtgaacgtgaattcg
tgagagaaagaggtcctcagagagtagcagctcacataactgggaccaga
ggaagaagcaacacattgtcttctccaaactccaagaatgaaaaggctct
gggccgcaaaataaactcctgggaatcatcaaggagtgggcattcattcc
tgagcaacttgcacttgaggaatggtgaactggtcatccatgaaaaaggg
attttactacatctattcccaaacatactttcgatttcaggaggaaataa
aagaaaacacaaagaacgacaaacaaatggtccaatatatttacaaatac
acaagttatcctgaccctatattgttgatgaaaagtgctagaaatagttg
ttggtctaaagatgcagaatatggactctattccatctatcaagggggaa
tatttgagcttaaggaaaatgacagaatttttgtttctgtaacaaatgag
cacttgatagacatggaccatgaagccagttttttcggggccatttttag
ttggctaaggatcc.

Said nucleotide sequence includes a Xba I restriction site sequence: tctaga, a Kozak sequence: gccatgggt,

and a secretory signal peptide encoding region with the sequence:

(SEQ ID NO: 2)
cgtcgagggcgtctgctggagatcgccctgggatttaccgtgcttttagc
gtcctacacgagccatggggcggacgcc,

and a trimer stabilizing structure encoding region with the sequence:

(SEQ ID NO: 3)
cgtatgaaacagatcgaagacaaaattgaggagatccttagcaagattta
ccatatagaaaacgagatcgctcgtattaaaaagcttatcggtgaacgt,

and the TRAIL 114-281 amino acid fragment encoding region with the sequence:

(SEQ ID NO: 1)
gtgagagaaagaggtcctcagagagtagcagctcacataactgggaccag
aggaagaagcaacacattgtcttctccaaactccaagaatgaaaaggctc
tgggccgcaaaataaactcctgggaatcatcaaggagtgggcattcattc
ctgagcaacttgcacttgaggaatggtgaactggtcatccatgaaaaagg
gtttactacatctattcccaaacatactttcgatttcaggaggaaataaa
agaaaacacaaagaacgacaaacaaatggtccaatatatttacaaataca
caagttatcctgaccctatattgttgatgaaaagtgctagaaatagttgt
tggtctaaagatgcagaatatggactctattccatctatcaagggggaat
atttgagcttaaggaaaatgacagaatattgatctgtaacaaatgagcac
ttgatagacatggaccatgaagccagttttttcggggcctttttagttgg
c,

and a BamH I restriction site sequence: ggatcc.

(2) double-enzyme digestion of double-strand nucleotide sequence and agarose gel electrophoresis recovery

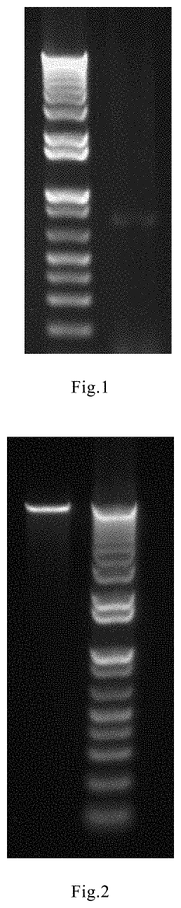

The synthesized double-strand DNA molecule was subjected to complete double-enzyme digestion with Xba I and BamH I (NEB Inc.) The digested product was analysis by agarose gel electrophoresis (as shown in FIG. 1). The 711 bp fragment was recovered with a gel-re

The synthesized double-stranded DNA molecules were fully double-digested with restriction endonucleases Xba I and BamH I (NEB Inc.). The digested products were analyzed by 1% agarose gel electrophoresis (Result shown in FIG. 1). The 711 bp nucleic acid fragment was purified by Qiagen gel recovery kit.

### EXAMPLE 2

### Preparation of a Secretory-Type Lentiviral Expression Vector pCDH-seTRAIL Encoding Secretory Fragment of TRAIL

(1) Double digestion of pCDH vector: The pCDH vector plasmid (System Biosciences Catalog: CD513B-1) were fully double-digested with restriction endonucleases Xba I and BamH I (NEB Inc.). The digested products were analyzed by 0.8% agarose gel electrophoresis (Result shown in FIG. 2). The ˜8000 bp nucleic acid fragment was purified by Qiagen gel recovery kit.

(2) Ligation and transformation of the digested product: The expression vector pCDH-seTRAIL was constructed by mixing the 711 bps encoding DNA fragment being digested and recovered in Example 1 and the 8,000 bps pCDH plasmid DNA digested with the same enzymes at a molar ratio of 5:1, using T4 DNA ligase (NEB) for ligation overnight at 16° C.

The ligation product was transformed into E. coli stbls competent cells (stbls competent cells were purchased from Genecopoeia), and the transformation was performed using the method described in F. Osbourne, R. Brent, R E Kingston, D D Moore, J G Seidman, J A Smith K. Straer, “Guidelines for Editing Molecular Biology”, John Wiley & Sons, New York, 1995, Third Edition, P 39-40. Transformants were achieved after screening with ampicillin resistance.

(3) Verification of the expression vector pCDH-seTRAIL: the selected positive transformants were cultured and plasmid DNA was extracted using plasmid DNA extraction method according to J. Sambrook, E F Ferric, T. Maniartis, Molecular Cloning: A Laboratory Manual, New York, Cold Spring Harbor Press, 2001, third edition, P 27-30. The vector was subjected to DNA sequencing for confirmation of the correct sequence.

### EXAMPLE 3

### Packaging and Transfection of Lentiviral Particles Encoding a Secretory Fragment of TRAIL

(1) Determination of concentration and purity of expression plasmid pCDH-seTRAIL and helper plasmids Using the third generation lentiviral packaging system, in addition to the expression plasmid pCDH-seTRAIL, the packaging plasmid pMDLg/pRRE (purchased from Addgene, Catalog: 12251) , the packaging plasmid pRSV-REV (purchased from Addgene, Catalog: 12253), the shell protein packaging plasmid pMD2G (purchased from Addgene, Catalog: 12259) were also needed to achieve the packaging of the virus particles. The purity and concentration of each plasmid DNA were analyzed using a NanoDrop spectrophotometer. The results are shown in Table 1.

TABLE 1
Concentration and purity of plasmids.

| plasmid | Conc. (ng/μl) | OD 260/280 | OD 260/230 |
|---|---|---|---|
| pCDH-seTRAIL | 1573 | 1.94 | 2.09 |
| blank plasmid pCDH | 1771 | 1.90 | 2.13 |
| pMDLg/pRRE | 360 | 1.87 | 2.27 |
| pRSV-REV | 395 | 1.85 | 2.15 |
| pMD2G | 376 | 1.98 | 2.36 |

(2) Co-transfection of HEK293 cell line with the plasmids

Mixing the following (every 15-cm culture plate):

| | | |
|---|---|---|
| pMD2G | 9 | μgμ |
| pMDLg/pRRE | 12.5 | μg |
| pRSV-REV | 6.25 | μg |
| pCDH-seTRAIL/pCDH | 32 | ug |
| (blank control) | | |
| 2.5M CaCl₂ | 125 | μl |
| 0.1 X TE buffer* | 1000 | μl |
| 2 X HBS^{#} | 1250 | μl |

*TE buffer (10 mM Tirs pH 8.0, 1 mM EDTA)
^{#}HBS (280 mM NaCl, 50 mM HEPES, 1.5 mM Na₂HPO₄•H₂O, 10 mM KCl, 12 mM Dextrose)

transfection:

A. One day before transfection, HEK293 cells (Clontech, Cat. No 632180) were inoculated into a 15-cm cell culture dish, the cell density was controlled to be about 80% on the inoculation day;

B. Each plasmid, CaCl₂ solution, TE buffer was thoroughly mixed in a 50 ml centrifuge tube, and the mixture was slowly added to another centrifuge tube in which the HBS solution was added;

C. Mixed at medium speed and added to each cell culture plate; the cells were incubated in a cell culture incubator;

D. 16 hours after transfection, the medium was carefully aspirated and replaced with DMEM medium containing 10% FBS;

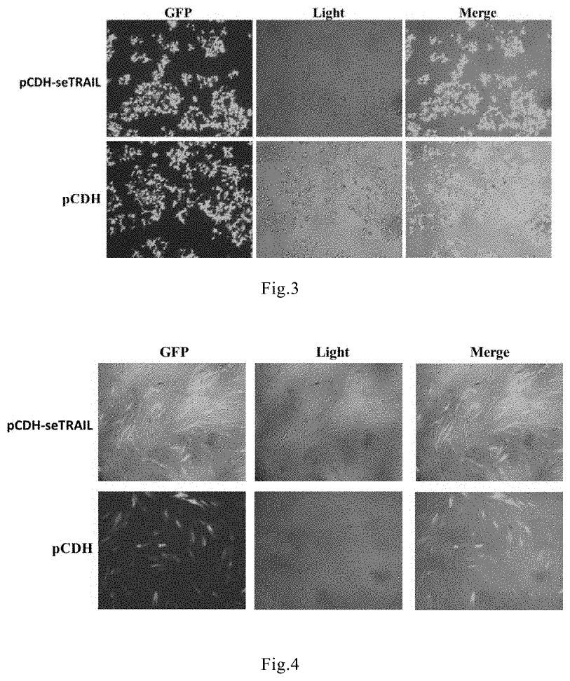

E. The cells were cultured for 48 hours. The fluorescence signal of the cells was observed by a fluorescence microscope. The result is shown in FIG. 3. FIG. 3 is a fluorescence micrograph of a HEK293 cell line co-transfected with the four plasmids. GFP: a photo under fluorescence, Light: a white light photo in the same field of view, Merge: an integrated photo of two fields of view.

(3) Virus particle collection

A. collected the above cell culture supernatant;

B. Being centrifuged at 800 g for 10 min at 4° C. , collected the supernatant;

C. The supernatant is passed through a 0.45 μm microporous membrane and the filtrate is retained;

D. For each four volumes of filtrate, add one volume of pre-cooled PEG-it Virus Precipitation Solution (System Biosciences Catalog: LV810A-1), mixed well, standed at 4° C. overnight, centrifuged at 1,500 g 30 min, virus particles precipitation were obtained;

E. Viral particles were resuspended as virus particle stocks with appropriate amount of TBS solution (800 mg NaCl, 20 mg KCl, 300 mg Tris base in 100 ml deionized water, pH 8.0), dispensed and stored at −80° C.

### EXAMPLE 4

### Titration of a Lentivirus Expressing Secretory Fragment of TRAIL

1. HEK293 cells were seeded in 96-well plates at a density of 4,000 cells per well, and placed in a cell culture incubator overnight;

2. Prepared 10-fold gradient dilutions of virus using in DMEM medium (10% FBS) containing 8 μg/ml of polybrene to dilute the virus particle stocks;

3. Carefully aspirated the medium in the 96-well plate and added each well the medium containing the gradient dilutions of the virus particles. Three Repeats for each concentration treatment group.

4. Incubated for 48 hours. HEK293 cells with green fluorescence signal were observed under a fluorescence microscope. The fluorescence signal of the lowest dilution was recorded (Table 2), and the titer of the virus particle stock was calculated as follows:

Titer (TU/μl)=1/10×10⁽ⁿ⁻¹⁾

n is the gradient dilution factor.

TABLE 2
Fluorescence signal detection result of the titration (✓ indicates
fluorescence, x indicates no fluorescence)

| dilution factor | 1 | 2 | 3 | 4 | 5 | 6 | 7 | 8 | 9 | 10 |
|---|---|---|---|---|---|---|---|---|---|---|
| pCDH-seTRAIL | ✓ | ✓ | ✓ | ✓ | ✓ | ✓ | ✓ | ✓ | x | x |
| pCDH (control) | ✓ | ✓ | ✓ | ✓ | ✓ | ✓ | ✓ | ✓ | x | x |

Calculated from the results as shown in Table 2 and the above formula

pCDH-seTRAIL lentivirus titration is 1×10⁶ TU/μl

pCDH (blank vector control) lentivirus titration is 1×10⁶ TU/μl

### EXAMPLE 5

### Transfection of Human Mesenchymal Stein Cells with a Lentivirus Expressing Secretory Fragment of TRAIL

1. Preparation of umbilical cord-derived human mesenchymal stem cell

Immediately after the delivery of the fetus, the umbilical cord was cut by routine obstetric ligature method; the umbilical cord was washed with saline, and then disinfected with medical alcohol; and the umbilical cord was placed in the umbilical cord preservation solution at a constant temperature of 2-8° C. The obtained umbilical cord was washed with 0.9% sodium chloride solution and repeated 2 to 3 times to remove blood stains. The entire umbilical cord was immersed in 75% ethanol for sterilization. Washing by sodium chloride solution repeatedly to remove residual ethanol. The umbilical cord was then cut into pieces of about 2 to 5 cm in length with a sterile surgical scissors, and removed congestion and clots in the small blood vessels of the umbilical cord. The white connective tissue between the amniotic membrane and the blood vessel was Wharton's jelly, which was torn off with a gingival sputum and then placed in a sterile dish. An appropriate amount of 0.9% sodium chloride solution was added to wash the colloid. Weighed Wharton's jelly was then cut into tissue pieces of 1 to 4 mm³ in size with a sterile tissue scissors, and 0.9% sodium chloride solution was added to wash it. The mixture was centrifuged at 800 to 900 g for 5 minutes. According to the weight of the colloid, appropriate medium was added, and the concentration of the tissue pieces was about 04-0.7 g/ml. After making the tissue pieces distributing evenly by pipetting, the tissue pieces were inoculated into a T75 flask, and the medium was added. The culture flask was placed flatly to make the tissue pieces being distributed as evenly as possible at the bottom surface. The flask was placed in a CO₂ incubator of constant temperature for cell culture. The Culture conditions: 37.0±0.5° C., carbon dioxide volume fraction 5.0±0.2%.

The first medium change: the tissue was cultured till the 5th to 7th day and the whole medium was changed. The unattached tissue pieces in the culture flask were combined with the old medium and transferred to a centrifuge tube, centrifuged 800-900 g for 5 min, the supernatant was removed, and the residual tissue pieces after centrifugation were added with the appropriate amount of fresh medium. After making the tissue distributing evenly by pipetting, the solution were inoculated into the original flask, and the medium was added for cell culture. The culture flask was placed flatly to make the tissue pieces being distributed as evenly as possible at the bottom surface. The flask was placed in a CO₂ incubator of constant temperature for continued cell culture.

The second medium change: the tissue was cultured till the 10th to 13th day and partial of the medium was changed. The flask was slightly tilted and half amount of the old medium was removed, and an equal amount of fresh medium was added. The culture was continued in the CO₂ incubator.

On the 14th to 18th day of the tissue pieces culture, when the area percentage of the cell clones reaches 70% to 80%, the cell culture was digested and harvested. The medium supernatant was removed and the cells were washed with 0.9% sodium chloride solution. Appropriate amount of digestive enzyme was added to the culture flask until the bottom of the culture flask was immerged. After standing for 1 min, the culture flask was inverted and observed under an microscope. The cells were round, and most of the adherent tissue blocks and cells fell off, and the digestion was then terminated (digestion time was less than 5 min). The cell suspension was transferred into a centrifuge tube, and a small amount of 0.9% sodium chloride solution was used to rinse the bottle wall. The washing liquid was transferred into a centrifuge tube and suspended for 30 seconds, filtered through a 100 μm sterile filter, and the filtrate was centrifuged, 300 g, 10 min. The washing supernatant was discarded and the cells were resuspended in 0.9% sodium chloride solution.

2. The cell culture was inoculate in cell culture dishes at a cell density of 60%, and incubated in a cell culture incubator overnight;

3. Carefully aspirated the medium from the cell culture dish, and carefully added DMEM medium (10% FBS) containing 8 μg/ml of polybrene.

4. pCDH-seTRAIL- or pCDH- (blank vector control) containing lentivirus were added to the cell culture respectively. The MOI (the ratio of virus TU to the number of target cells) was about 10. The cells were continued for culture for 24 hours in the cell culture incubator;

5. Carefully aspirated the medium in the cell culture dish; α-MEM medium containing 10% FBS was added to each cell culture dish and continued to culture for 48 hours;

6. MSC cells with green fluorescence signals were observed with fluorescence microscopy. Cell culture supernatants were collected. Results were shown in FIG. 4, GFP: a photo under fluorescence, Light: a white light photo in the same field of view, Merge: an integrated photo of two fields of view.

### EXAMPLE 6

### Activity of MSC Cells Infected By Lentiviral Vector to Express and Secrete TRAIL

The collected cell culture supernatant was analyzed for the TRAIL content by using Abcam's TRAIL Human ELISA Kit (Catalog: ab46074) according to the kit's instructions. The recombinant human TRAIL provided in the kit was used to provide a standard curve.

The results of the measurement are shown in FIG. 5:

The concentration of TRAIL in the supernatant of MSC medium infected with lentivirus pCDH-seTRAIL was approximately 634.93 pg/ml.

The concentration of TRAIL in the supernatant of MSC medium infected with the control vector lentiviral pCDH was approximately 32.96 pg/ml. This amount is within the test error and the instrument reading background error range.

### EXAMPLE 7

### Inhibition of Human Glioma Cell Line U87 Cells By MSC Cells Secreting TRAIL

1. The collected cell culture supernatant of the MSC infected with the virus particles was centrifuged at 1,000 rpm for 10 minutes to remove the remaining cells, and the supernatant was aspirated as a conditioned medium;

2. The conditioned medium was 2-fold concentration gradient diluted with α-MEM medium (comprising 10% FBS); medium of concentrations of 1, 0.5, 0.25, and 0.125-fold dilution were prepared;

3. Human glioma cell line U87 cells (purchased from ATCC, Cat. No. HTB-14) cultured in α-MEM medium containing 10% FBS were seeded in 96-well plates at a density of 3,000 cells per well. Incubated overnight in a cell culture incubator;

4. Carefully removed the culture supernatant from each well;

5. Added 200 ul of each concentration of conditioned medium to each well, and set a blank control well (α-MEM medium containing 10% FBS was added) and continued to culture for 72 hours;

6. Added 20 ul of preset MTS solution (Promega) to each well and continued to culture for 3 hours.

7. Measured the absorbance of each well using a Thermo Scientific Mulitiskan microplate reader at a wavelength of 490 nm and a reference wavelength of 690 nm;

8. Calculated the tumor inhibition rate according to the following formula:

tumor inhibition rate (%)=(blank control absorbance value−conditioned medium treatment absorbance value)/blank control absorbance value×100

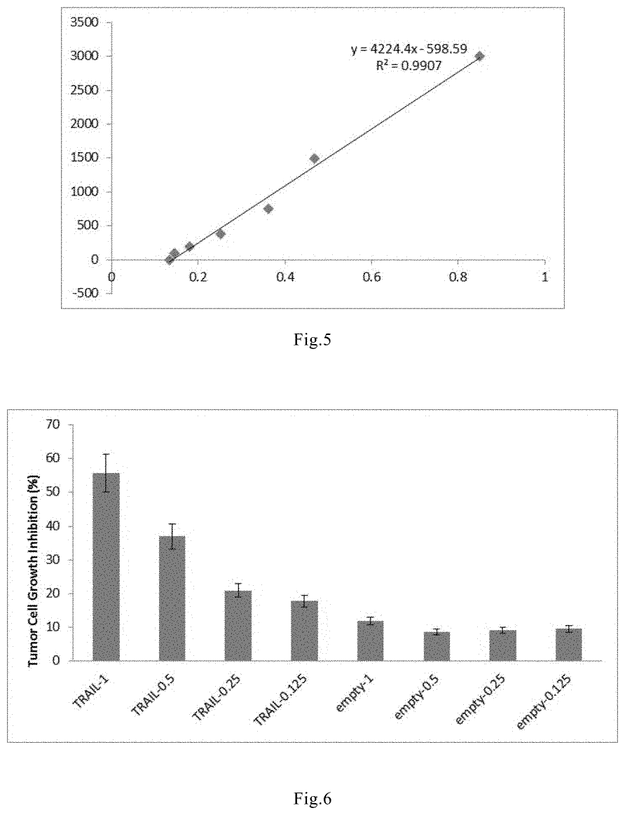

The result was shown in FIG. 6: after the lentiviral vector expressing the secretory TRAIL of the present invention infected MSCs, MSCs expressed and secreted a large amount of biologically active TRAIL into the cell culture medium, and significantly inhibited the growth of U87 cells.

### EXAMPLE 8

### Stability of TRAIL Secreted By MSCs Infected with Lentiviral Vector

1. The conditioned medium supernatant of the MSCs infected with the lentivirus pCDH-seTRAIL obtained in Example 6 (the TRAIL concentration in the supernatant was detected as: 634.93 pg/ml) was set as the object of test;

The conditioned medium supernatant of the MSCs infected with the control vector lentiviral pCDH was set as the first control medium;

The fresh medium supernatant was set as the second control medium (the concentration of TRAIL in the supernatant was detected to be around 0);

Commercially available recombinant human rhTRAIL protein (Peprotech, Cat. No. 310-04) was used as reference. The commercially available recombinant human rhTRAIL protein was dissolved in the first control medium and the second control medium, respectively, to a final concentration of 650 pg/ml.

The conditioned medium of MSCs infected with lentivirus pCDH-seTRAIL, the supernatant of the first control medium in which rhTRAIL was dissolved, and the supernatant of the second control medium in which rhTRAIL was dissolved were placed in a 37° C. warm bath. A small amount of supernatant samples were taken therefrom at different times (0, 3, 6, 12, 24, 48 h) and stored in a refrigerator at −70° C.

After samples at all time points were collected, the TRAIL content of each sample was determined by the ELISA method mentioned in Example 6.

The protein content at 0 h of each treatment group was 100%, and the residual rate of TRAIL at each time point of each treatment group was calculated according to the following formula:

Residual rate=(TRAIL concentration at each time point/TRAIL concentration at 0 h time point)×100%

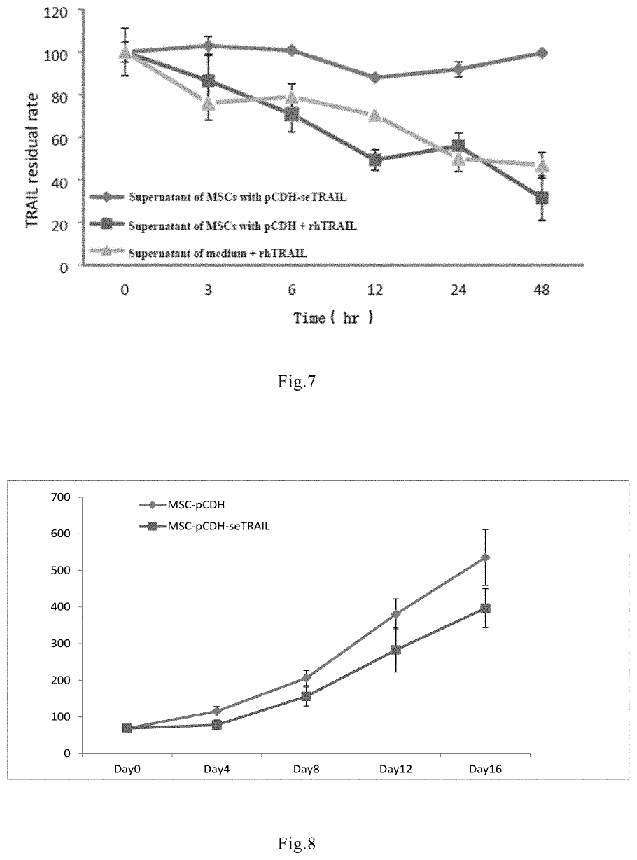

The results are shown in FIG. 7. The results proved that was confirmed that the secretory TRAIL fragment produced and secreted by the MSC cells carrying the lentiviral vector the present invention has higher stability than the commercially available recombinant human rhTRAIL protein.

### EXAMPLE 9

### Inhibition of Human Glioma Cells in Nude Mice By MSC Cells Secreting TRAIL in an Animal Model

The experimental animals were male nude mice of 4-6 weeks (BALB/C nude mice, Chinese University of Hong Kong Experimental Animal Center). Breeding under standard experimental conditions: 12 hours light-12 hours dark cycle, free access to water and food.

MSCs infected with lentivirus pCDH-seTRAIL, MSCs infected with control vector lentivirus pCDH, and human glioma cell line U87 cells were cultured respectively in α-MEM medium containing 10% FBS, under 37° C. , 5% CO₂ saturated humidity conditions. Four days after the animals were adapted to the culture environment, U87 cells were injected subcutaneously into nude mice, and each animal was inoculated with U87 cells into two positions, which were the left side of the back and the symmetry site at the right side. The inoculation amount of U87 was 2×10⁶ cells and the injection volume was 100 ul. The entire operation is done in a Bechtop bench. The health status of the mice and the growth of the tumor were monitored after the completion of the inoculation. After all the mice developed obvious solid tumor, 8 mice with similar tumor size on the left and right sides (the diameter of the tumor was about 5 mm using a vernier caliper for measurement) were selected according to the tumor volume of the animals. 2×10⁶ MSCs infected with lentivirus pCDH-seTRAIL were injected into the left tumor, while 2×10⁶ MSCs infected with the vector lentivirus pCDH were injected into the right tumor, and the injection volume were 100 ul. The tumor size was measured with a vernier caliper every 4 days, and the tumor volume was calculated according to the following formula: tumor volume (mm³)=1/2 ab², a is long diameter of the tumor, and b is short diameter of the tumor.

Data analysis between groups was statistically analyzed using ANOVA statistical methods, and P<0.05 was considered to be significantly different.

FIG. 8 shows the average tumor volume measurement results of the two groups (volume unit mm³)

After injection of lentiviral-infected MSCs, tumor volume began to differentiate between the two groups. The tumor volume of the MSCs injected with lentivirus pCDH-seTRAIL was gradually smaller than that of the control group injected with MSCs infected with the control vector pCDH. And on the 12th day and the 16th day, there was a significant difference (P<0.05). The experimental results demonstrate that the MSC cells of the present invention infected with a lentiviral vector bearing a secretory TRAIL encoding insert have a significant therapeutic effect on human glioma in vivo.

The inventors of the present invention have unexpectedly found that umbilical cord mesenchymal stein cells prepared by the method of the present invention are effective in inhibiting brain tumors, particularly gliomas, in vivo. One of the reasons for this unexpected discovery is the construct encoding a secretory TRAIL soluble fragment of the present invention and a lentiviral expression vector containing said construct.

In said construct of the present invention, an amino acid sequence which can promote the formation of a trimer structure of TRAIL is added to the encoding region of the soluble fragment of the TRAIL, which contributes to the stability and activity of the expressed TRAIL soluble fragment. In addition, the secretory TRAIL soluble fragment expression vector of the present invention has a highly efficient secretory signal peptide sequence at the upstream region of the soluble TRAIL fragment encoding sequence, so that the TRAIL fragment cloned downstream can be expressed and secreted into the extracellular medium, which significantly increases the efficiency of protein expression. The stability of the soluble fragment of the produced TRAIL is unexpectedly increased. In particular, by using a lentiviral vector, the insert encoding a soluble fragment of secretory TRAIL can be stably integrated into the genome of a host cell, which is not easily lost, and reduce the risk of insertional tumor formation, thus is more safe and reliable.

The practice of the present invention will employ, unless otherwise indicated, conventional techniques of biotechnology, organic chemistry, inorganic chemistry, and the like. Other aspects and modifications within the scope of the invention will be apparent to those skilled in the art. Variations and modifications are possible in light of the teachings of the invention and are therefore within the scope of the invention. All patents, patent applications, and scientific papers referred to herein are hereby incorporated by reference.

1. An isolated construct, wherein the construct comprises (1) a secretory signal peptide encoding region; (2) a TRAIL trimer stabilizing structure encoding region; and (3) a TRAIL fragment encoding region, wherein the TRAIL fragment has an amino acid sequence identical or substantially identical to the amino acid sequence of (a) or (b) below:
(a) amino acid residues 114 to 281 of TRAIL;
(b) having at least 90% identity with the amino acid sequence of (a), and having TRAIL activity.

2. The construct of claim 1, wherein the secretory signal peptide is a human fibrillin secretory signal peptide, a human growth hormone secretory signal peptide, a human immunoglobulin signal peptide, or a human interleukin 2 signal peptide.

3. The construct of claim 1, wherein the TRAIL trimer stabilizing structure encoding region is an isoleucine zipper structure or a leucine zipper structure.

4. The construct of claim 1, wherein the TRAIL fragment encoding region has the sequence of SEQ ID NO:1.

5. The construct of claim 1, which has the sequence of SEQ ID NO:4.

6. A vector comprising the construct of claim 1, wherein the vector is an animal cell expression vector.

7. A mammalian cell whose genome is integrated with the construct of claim 1 and which expresses and secretes the TRAIL or a fragment thereof.

8. The mammalian cell of claim 7, which is a mesenchymal stem cell, a bone marrow mesenchymal stem cell, or a fat mesenchymal stem cell.

9. The mammalian cell of claim 7, which is prepared by the method described below:
a. providing a construct according to claim 1;
b. Preparing a vector comprising the construct, wherein the vector is an animal cell expression vector;
c. Infecting a mammalian cell with the vector.

10. A pharmaceutical composition for treating a brain tumor, such as a glioma, which comprises the construct of claim 1 or the vector of claim 6 or the mammalian cell of claim 7.

11. Use of the construct of claim 1 or the vector of claim 6 or the mammalian cell of claim 7 or the pharmaceutical composition of claim 10 for treating a brain tumor.

12. Use of the construct of claim 1 or the vector of claim 6 or the mammalian cell of claim 7 or the pharmaceutical composition of claim 10 in the manufacturing of a medicament for treating a brain tumor.

13. The construct of claim 2, wherein the secretory signal peptide is a human fibrillin secretory signal peptide and has the sequence of SEQ ID NO: 2.

14. The construct of claim 3, wherein the TRAIL trimer stabilizing structure encoding region has the sequence of SEQ ID NO: 3.

15. The vector of claim 6, wherein the vector is a lentiviral vector.

16. A mammalian cell of claim 7, wherein said mammalian cell is a stem cell of human, mouse, rat, pig or monkey.

17. A mammalian cell of claim 8, wherein said mammalian cell is an umbilical cord mesenchymal stem cell.

18. Use of claim 11, wherein said brain tumor is a glioma.

19. Use of claim 12, wherein said brain tumor is a glioma.


SEQUENCE LISTING (5 sequences)

SEQ ID NO: 1 - DNA, 504 bp, artificial sequence
  Description of Artificial Sequence Synthetic
gtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggc

SEQ ID NO: 2 - DNA, 78 bp, artificial sequence
  Description of Artificial Sequence Synthetic
cgtcgagggcgtctgctggagatcgccctgggatttaccgtgcttttagcgtcctacacgagccatggggcggacgcc

SEQ ID NO: 3 - DNA, 99 bp, artificial sequence
  Description of Artificial Sequence Synthetic
cgtatgaaacagatcgaagacaaaattgaggagatccttagcaagatttaccatatagaaaacgagatcgctcgtattaaaaagcttatcggtgaacgt

SEQ ID NO: 4 - DNA, 711 bp, artificial sequence
  Description of Artificial Sequence Synthetic
tctagagccatgggtcgtcgagggcgtctgctggagatcgccctgggatttaccgtgcttttagcgtcctacacgagccatggggcggacgcccgtatgaaacagatcgaagacaaaattgaggagatccttagcaagatttaccatatagaaaacgagatcgctcgtattaaaaagcttatcggtgaacgtgaattcgtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaaggatcc

SEQ ID NO: 5 - protein, 281 aa, Homo sapiens [sequence omitted]